(19) United States
(12) Patent Application Publication
(10) Pub. No.: US 2015/0025419 A1
(43) Pub. Date: Jan. 22, 2015

(54) INTEGRATED TRIAD SYSTEM FOR PROVIDING ADVANCED ASSESSMENT METHODS, CORRECTIVE AND PERFORMANCE EXERCISE TRAINING, AND INTEGRATING SPECIALIZED MANUAL THERAPIES

(71) Applicant: Everett Aaberg, Dallas, TX (US)

(72) Inventor: Everett Aaberg, Dallas, TX (US)

(21) Appl. No.: 13/945,135

(22) Filed: Jul. 18, 2013

(51) Int. Cl. A61B 5/00; A61B 5/11

(52) U.S. Cl. CPC A61B 5/7282; A61B 5/1121; A61B 5/1116; A61B 5/112
USPC 600/595

(57) ABSTRACT

The Ortho-Kinetics System is a biomechanically based system that, through systematic assessment, analysis, and evaluations, one can identify a person's anomalies such as postural deviations, joint limitations, neuromuscular imbalances, and movement compensations. This system makes use of a database which stores expert-derived information to guide the practitioner in the administration and performance of advanced assessment methods and programming process, corrective and performance exercise training, along with integration of specialized manual therapies.

### FIELD OF THE INVENTION

This invention relates to the field of human health and fitness and, in particular, to an integrated triad system which implements an automated, expert-system driven, three step process: test the client to automatically identify anomalies, treat the client with an expert-system derived plan of action to resolve the underlying problem, and train the client to achieve optimal human performance.

### BACKGROUND OF THE INVENTION

It is a problem in the field of human health and fitness to provide the client with an effective and complete remediation program. This is due to the fact that the personal training industry is presently unregulated; there are no federal or state laws that require trainers to have any licensing, state registration, or obtain any specific minimum formal education, experience, or training prior to offering their services. Although there are hundreds of “Personal Trainer” and “Fitness Instructor” certifications available, the majority of these are offered on-line; and the handful of Personal Training Certifications that do require some class time are only two- to four-day courses that offer little or no hands-on training nor have set any formal educational prerequisites.

While the effectiveness of specific manual therapies and tissue treatments has proven to be of great value for treating injuries, improving movement ability, and enhancing physical performance for people of all ability levels and at all age ranges, these are but one segment of the solution to the problems of a client. Techniques such as “Active Release Technique®” are rapidly growing in demand and popularity. Such manual therapies are focused primarily on increasing muscle and joint function and also can help to reduce pain and potential injury associated with exercise and sports participation. However, these techniques are presently not integrated with a comprehensive assessment and programming process nor are these techniques performed in conjunction with any specific “Corrective” and/or “Performance”-based exercise selections and techniques. Manual therapies typically are not effective long term without a tailored “Corrective and Performance Exercise Program” systematically selected from advanced Assessment Methods that will reinforce treatments, retain range of motion, correct muscular imbalances, reduce or eliminate compensation, and re-educate the entire Neuromuscular System. Such assessment methods and specific exercises and techniques are critical for restoring function and optimal performance.

### BRIEF SUMMARY OF THE INVENTION

The above-described problems are solved and a technical advance achieved by the present Integrated Triad System For Providing Advanced Assessment Methods, Corrective And Performance Exercise Training, And Integrating Specialized Manual Therapies (termed “Ortho-Kinetics System” herein), which is an automated and comprehensive computerized system that combines advanced assessment methods, a programming process, corrective and performance exercises and techniques, along with specialized manual therapies, when needed, that are designed to restore function and develop optimal human performance.

The Ortho-Kinetics System is a bio-mechanically based system that recognizes the interdependency of the anatomical structural, muscular, and neurological systems; and that they have logical, purposeful, functional, and definitive integration. Therefore, through the use of systematic assessment, analysis, and evaluation, one can identify faulty structural, muscular, or neurological dysfunction. In addition, the logical selection and precise application of specific training and treating methods and techniques address the identified dysfunction and correct any anomalies. As such, the logic of the Ortho-Kinetics System has been translated to a computerized system, which is based on the interdependence of the three primary movement systems of the body: the passive-skeletal system, the active-muscular system, and the control-sensory-motor system. Due to the fact that human movement, though extremely complex, is reliant upon the interdependence of these three systems and is governed by the laws of physics and the associated mechanical principles that always remain objective, there is a level of predictability as to cause and effect to any movement deficits, joint misalignments, postural deviations, neuromuscular imbalances, and compensation that are identified within the Ortho-Kinetics System during the “testing” phase. As such, a numerical value for each joint deviation in each plane can be assigned to create a mathematical formula to derive all combinations that the Ortho-Kinetics System can generate. This in turn creates an extensive flow chart format for the development of the decision tree upon which the Ortho-Kinetics System is based.

The Ortho-Kinetics System creates a numerical value that represents the specific “Postural Pattern” derived from the cumulated joint deviations at each joint included in the decision process and relative function in all three planes as discovered within the testing process. This automated process identifies the specific Postural Pattern of the current 23,265 possible Postural Patterns. This identified Postural Pattern suggests, guides, and helps in the selection of the specific training and treatment remedies that the certified Trainer/Therapist (termed “Practitioner” herein) utilizes to create and administer a corrective- and performance-based exercise program and integrated treatment plan for a Practitioner's patients and athletes. Further, the Practitioner continually utilizes the Ortho-Kinetics System as a tool for reassessing, to provide direction, offer suggestions, log exercise, record treatment, track changes, mark progressions, and help to communicate with the client, patient, or athlete.

### BRIEF DESCRIPTION OF THE DRAWINGS

FIG. 1 illustrates, in block diagram form, the overall architecture of a computerized implementation of the present Ortho-Kinetics System;

FIG. 2 illustrates a typical menu that is used in the operation of the Ortho-Kinetics System;

FIG. 3 illustrates, in flow diagram form, the operation of the Ortho-Kinetics System Assessment and Programming Process;

FIGS. 4A-4D illustrate examples of charts produced by the Ortho-Kinetics System to guide a practitioner in the use of the Ortho-Kinetics Assessment and Programming Process in identifying specific postural deviations;

FIGS. 5A and 5B illustrate assessment screens produced by the Ortho-Kinetics System within the Ortho-Kinetics Assessment and Programming Process to guide a practitioner in determining a client's Head Rotation;

FIGS. 6A and 6B illustrate assessment screens produced by the Ortho-Kinetics System within the Ortho-Kinetics Assessment and Programming Process to guide a practitioner in determining a client's shoulder mobility;

FIGS. 7A-7C illustrate assessment screens produced by the Ortho-Kinetics System within the Ortho-Kinetics Assessment and Programming Process to guide a practitioner in determining a client's trunk mobility;

FIGS. 8A and 8B illustrate assessment screens produced by the Ortho-Kinetics System within the Ortho-Kinetics Assessment and Programming Process to guide a practitioner in determining a client's pelvic mobility;

FIGS. 9A and 9B illustrate examples of charts produced by the Ortho-Kinetics System to guide a practitioner in the use of the Ortho-Kinetics Assessment and Programming Process in identifying specific Range of Motion Limitations;

FIGS. 10A and 10B illustrate an example of charts produced by the Ortho-Kinetics System to guide a practitioner in the use of the Ortho-Kinetics Assessment and Programming Process in identifying specific muscular imbalances;

FIG. 11 illustrates an example of charts produced by the Ortho-Kinetics System within the Ortho-Kinetics Assessment and Programming Process in reconciling conflicts between the Postural Assessment and the Joint Analysis processes;

FIG. 12 illustrates, in flow diagram form, the operation of the Ortho-Kinetics System utilized for basic logic for decision making in the Ortho-Kinetics Assessment and Programming Process for integrating specific Manual Therapies and Treatments;

FIG. 13 illustrates an example of a chart produced by the Ortho-Kinetics System to guide a practitioner in the use of the Ortho-Kinetics Assessment and Programming Process in identifying treatment suggestions;

FIG. 14 illustrates, in flow diagram form, the operation of the Ortho-Kinetics System as related to the Corrective and Performance Training Process;

FIG. 15 illustrates an example of a chart produced by the Ortho-Kinetics System to guide a practitioner in the use of the Ortho-Kinetics Assessment and Programming Process in identifying selection of the corrective and performance exercise; and

FIG. 16 illustrates the structure and operation of the expert system embodied in the Ortho-Kinetics System.

### DETAILED DESCRIPTION OF THE INVENTION

The present Ortho-Kinetics System is designed for use by trained and certified practitioners who have completed a course of study of the relevant attributes of the human body. The Ortho-Kinetics System has been developed and is now being utilized in practice and has a proven track record of success. It is performed through the use of an interactive computerized system that receives critical patient data that is input by a trained practitioner and which is obtained in a discovery phase where it is derived from certain Advanced Assessments pursuant to the Ortho-Kinetics System. The Ortho-Kinetics System then helps to combine and compare data and in turn generates a series of automated suggestions and directions for integration of specific corrective and performance exercise selections, overall programming options, further testing, logical treatment strategies, and suggested application of specialized manual therapies and overall treatment plans for the client. The Ortho-Kinetics System also tracks and provides automated analysis, detailed reports, and specific feedback to the practitioner thereby to enable them to evaluate the system's effectiveness at helping the client to realign their posture deviations, correct individual and systemic muscular imbalances, decrease compensation, reduce pain, increase range of motion, create stability, develop strength, improve speed, maximize power, and enhance their overall physical performance in order to optimize the client's physical potential and promote abundant health. Better movement ability for a client provides for better exercise, which leads to improved performance, enhanced aesthetics, and an increased level of overall health. Briefly stated, the Ortho-Kinetics System provides an automated set of biomechanical and neuromuscular assessments that are used to generate tailored programs and plans that integrate specialized manual therapies, corrective and performance exercise movements and techniques, and precision training to enable the practitioner to enable their clients to obtain better function, fitness, and performance, and to achieve results beyond what any exercise or treatment modality can independently deliver.

Effective and efficient movement of the human body relies on a precise, interdependent relationship among three architectural systems: the passive-skeletal system, the active-muscular system, and the control-sensory motor system. By understanding this true interdependent relationship, and being able to identify anomalies in the performance of the human body that relate to specific faulty status or interactions of these primary movement systems, a practitioner, trained in the use of the Ortho-Kinetics System, is able to identify the root cause of a client's problem and identify remedial action(s) which restore the proper balance and function among these primary movement systems. This process is implemented by the use of the Assessment and Programming Process of the Ortho-Kinetics System, which can be performed manually by a trained and certified Ortho-Kinetics Practitioner with the use of specialized assessment reports, programming workbooks, and exercise manuals, or their use of the on-line Ortho-Kinetics Computerized Assessment, Training, and Therapy Application (CATT Application) and its associated database, which stores expert-derived information, guides the practitioner in the performance of the following processes, which comprise a comprehensive system, that combines the Integrated Triad of: (1) advanced assessment methods (termed “Ortho-Kinetics Assessment and Programming Process” herein), (2) corrective and performance exercise, techniques and, professional training (termed “Ortho-Kinetics Corrective and Performance Training” herein), along with (3) selective use of specialized manual therapies (termed “Ortho-Kinetics Integrated Manual Therapy” herein). The Ortho-Kinetics System produces and utilizes an extensive database of client and patient assessment results; programming information and history; thousands of specific functional, fitness, muscle building, corrective, and performance exercises, techniques, modifications, and progressions that, with access to its contents and use of the data, can be implemented in various forms. It can be realized as an expert system resident on a computer system, which automatically responds to data input by a certified practitioner by presenting information to the certified practitioner to guide the selection of tests, therapies, and corrective exercises. Alternatively, the contents of the database can be realized in part in the form of a tangible product, such as a book and/or the charts illustrated herein which the certified practitioner can use to guide them through some of the steps of the Ortho-Kinetics System to execute the process. The three primary components, as summarized below, and the programming process of integration of these as interdependent and synergistic parts, is collectively the intellectual property that drives the Ortho-Kinetics System and, therefore, also comprises the decision tree and operational decision making for the CATT Application.

### First Component—The Ortho-Kinetics Assessment and Programming Process

Utilizing the knowledge and skills learned through certification courses, and armed with Ortho-Kinetics Manuals and/or the CATT Application, the Ortho-Kinetics Practitioner:

(1) Administers the Ortho-Kinetics “Assessment and Programming Process” that is comprised of the “Postural Pattern Assessment,” “Joint Bias Analysis,” “Functional Movement Evaluation,” and “Neuromuscular Capacity Testing” to the client to systematically identify, record, and compare anomalies such as Postural Deviations, Joint Limitations, Movement Compensation, and Neuromuscular Imbalances. Results of the Assessment and Programming Process are used to generate recommendations of specific corrective and performance exercises and potentially specialized manual therapies to the Practitioner to design customized corrective and performance exercise programs and treatment plans for the client, patient, or athlete.

(2) Makes final programming adjustments, then provides precision training of the specific traditional, contemporary, and corrective and performance exercises along with the techniques, modifications, and progressions as taught in the Ortho-Kinetics courses in order to help realign posture, restore movement ability, regain neuromuscular balance, and improve overall function of the client, patient, or athlete.

(3) To better or more quickly achieve these goals, enhance results, or provide for optimal performance, the Ortho-Kinetics System also directs the Practitioner with integration of specialized manual therapies.

The Ortho-Kinetics System utilizes both a manual process and a computerized Programming Process that not only selects specific corrective and performance exercises, but also guides the selection and integration of Specific Manual Therapies and Treatment Techniques. Some well-known therapies and techniques, such as “Active Release Techniques®,” “Myofacial Release,” “Muscle Activation Techniques,” and “Proprioceptive Neuromuscular Facilitation” techniques have been successfully implemented with the use of this system. Additionally, new manual therapies developed within the Ortho-Kinetics System such as “Ortho-Kinetic Proprioceptive Stimulation” and “Ortho-Metrics” also have been effectively integrated as a synergistic component of the Ortho-Kinetics System.

The Ortho-Kinetics Assessment and Programming Process identifies specific joint or muscular issues that would benefit from application of such specialized soft tissue treatment and manual therapies. The system's programming process directs the Practitioner to use such treatments and manual therapies and guides them to efficiently integrate them in conjunction with the corrective and performance exercise program. The system can utilize the same programming process for the implementation of active or passive stretching techniques, isometric techniques, and even plyometric exercises for addressing specific issues, progressing function, and enhancing performance.

The Ortho-Kinetics Assessment and Programming Process is a comprehensive biomechanically-based process of combining advanced assessment methods such as the Postural Pattern Assessment, a detailed Joint Bias Analysis, a Functional Movement Evaluation, and Neuromuscular Capacity Testing. The Ortho-Kinetics Assessment and Programming Process identifies postural deviations, discovers individual and sub-system muscular imbalances, and unveils movement limitations that collectively create compensation, cause pain, inhibit performance, and deteriorate overall health. The Assessment includes both a manual and a computerized process that directs the Trainer/Therapist toward effective selection of corrective and performance exercises, selection and use of specialized manual therapies, and guides the design of the overall program based on the combined results of the various assessments.

This comprehensive assessment process begins with the practitioner performing an in-depth lifestyle interview and health history of the client that is designed to establish fitness objectives, set realistic performance goals, and determine any special needs or challenges that may interfere with obtaining optimal results. The computerized Ortho-Kinetics assessment process then progresses on to include several methods of advanced assessment such as the Postural Pattern Assessment designed to identify the combination of deviations that relate to potential past injuries, muscular imbalances, and movement compensations. This is followed by the Joint Bias Analysis which is a precise joint-by-joint range-of-motion analysis that identifies all joint movement limitations, and then proceeds to Neuromuscular Capacity Testing which discovers the collection of individual and subsystem muscular weakness associated with the discovered Postural Deviations and Joint Bias. This cumulative detailed information then is correlated with and compared to the Functional Movement Evaluation that collectively helps to generate final mapping of postural deviations, joint bias, and muscular imbalances that identifies the specific Postural Deviation Pattern from the 23,265 possible Postural Deviation Patterns. Utilizing this data, the system then drives selection of the corrective and performance exercises and potential selection of specialized manual therapies. The Ortho-Kinetics System then automatically guides design and development of the tailored training program and treatment plan. The selected exercises are sequenced into single training routines which are arranged to comprise overall training programs that systematically manipulate all acute training variables such as intensity, volume, tempo, and recovery during different phases of the program. This overall comprehensive system will empower the trainer to help their clients address their individual needs, restore function, or reach their loftiest fitness and/or athletic goals and achieve optimal performance. The tracking, analysis, and comparison of the various assessments, as well as the prioritization of needs, integration of goals, and the systematic development of “Current Programming Theory,” is an exclusive Ortho-Kinetics Process taught in the Ortho-Kinetics Courses. The programming process can be done either manually through use of specially designed workbooks, but now also more efficiently through the Ortho-Kinetics Computerized Assessment, Training, and Therapy, (CATT) Online Application.

### Second Component—Corrective and Performance Exercise Training, Techniques and Progressions

Most any functional exercise or sports-specific motion can be easily integrated into the Ortho-Kinetics System once the movement is analyzed, classified, possibly modified, and then correlated to the movement needs identified in the Comprehensive Assessment Process summarized above. Thousands of such exercises are already contained within the database, along with specific corrective and performance exercises developed from in-depth study and synergistic application of anatomical design, motor programming, spinal mechanics, functional movement patterns, and gait biomechanics. Specific recommendations of these isolated and compound corrective and performance exercise movements derived from the assessment process noted above then are selected by the trainer/therapist, sequenced into training routines, then grouped together to form overall training programs, through the programming process.

### Third Component—Integration of Specialized Manual Therapy

The Ortho-Kinetics Process for integration of Specialized Manual Therapies entails the use of a single or combination of specialized manual therapies. Some of these therapies and techniques have been in existence for some time and have an established track record of success such as such as “Active Release Techniques®,” “Myofacial Release,” “Muscle Activation Techniques,” and “Proprioceptive Neuromuscular Facilitation” techniques. Such therapies have been effectively integrated along with the Ortho-Kinetics System by trained and certified Trainers who have been licensed to provide such therapies. In such cases, beneficial results were often achieved that application of the treatments or training alone would not have accomplished. Additionally, new manual therapies developed within the Ortho-Kinetics system such as “Ortho-Kinetic Proprioceptive Stimulation” and “Ortho-Metrics” have also been recently integrated as a synergistic component of the Ortho-Kinetics System and have also showed increased results for addressing client needs and accomplishing their goals than training or therapy could have produced individually. Selection, application, and integration of such manual therapies is achieved through the Ortho-Kinetics Assessment and Programming Process much in the same manner as the corrective and performance exercise training is accomplished. The information and data derived through the assessment process such as the client's, patient's, or athlete's postural deviations, neuromuscular imbalances, joint bias, and movement compensations will in turn drive logical recommendations as to specific individual muscles and subsystems that may benefit from certain soft tissue therapy or treatment techniques. Most all manual therapies can be classified as either “release” type techniques or “stimulation” techniques. Release therapies such as Active Release Techniques® (ART®) help the muscles lengthen better and more easily, allow for improved joint motion, and reduce inhibition of the antagonist muscles that helps them to contract more efficiently, improve overall movement performance, and often reduce pain. Therapies or techniques designed to “stimulate” a muscle help to facilitate neural communication, improve production of greater muscular force, provide joint stability, and also improve overall movement performance and can reduce pain. Through the Ortho-Kinetics System, Trainers and Therapists who are trained and licensed to utilize such therapies can better track, organize, and analyze data, while administrating assessments, designing programs, developing treatment plans, and evaluating client progressions when integrating manual therapies. This improves efficiency, whether providing a specific form of therapy or treatment independently or synergistically with others, and/or also integrated with the Ortho-Kinetics Corrective and Performance Training Exercises, as well as stretching, isometrics or other forms of exercise and therapy.

### Ortho-Kinetics Computerized Assessment, Training, and Therapy (CATT) Online Application

The Ortho-Kinetics System is a comprehensive automated expert-driven system that combines advanced assessment methods, corrective exercises and performance training, along with specialized manual therapies designed to synergistically develop optimal human performance. The Ortho-Kinetics System can be performed manually by trained and certified practitioners with the use of assessment logs, programming workbooks, and manuals containing numerous traditional, contemporary, and corrective and performance exercises. This system also has been developed to be used as a more automated and more efficient system known as the Ortho-Kinetics Computerized Assessment, Training, and Therapy (CATT) online Application. The CATT Application helps the practitioner work with a client to realign the client's posture, correct muscular imbalances, decrease compensation, reduce pain, increase range of motion, create stability, develop strength, improve speed, and maximize power in order to reach the client's physical potential and promote abundant health. Better movement ability provides for better exercise which leads to improved performance, enhanced aesthetics, and an increased level of overall health. The Ortho-Kinetics CATT Application, comprised of biomechanical and neuromuscular assessments, specialized manual therapies, and corrective exercise and functional fitness and performance training, achieves results beyond what any client exercise modality can deliver.

### Operation of the Ortho-Kinetics CATT Application

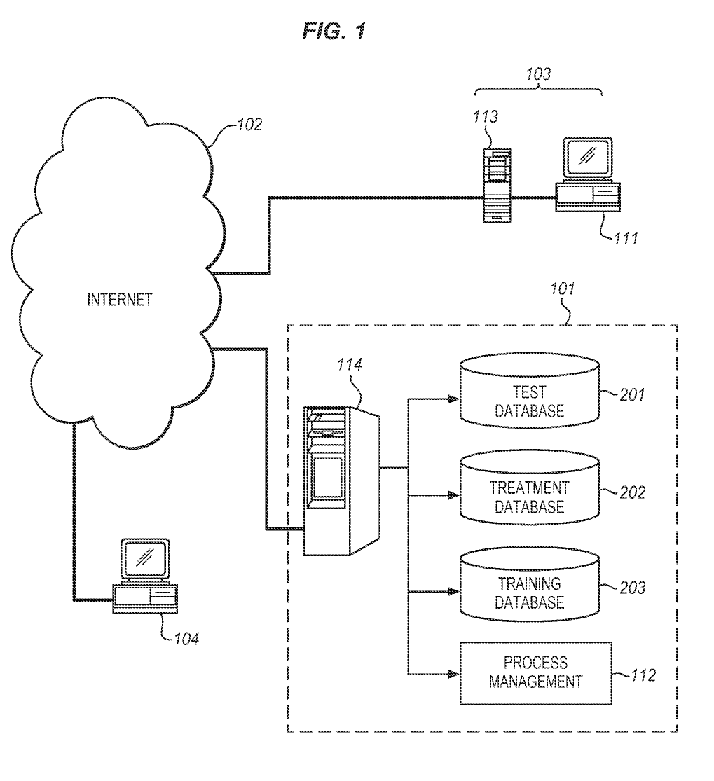

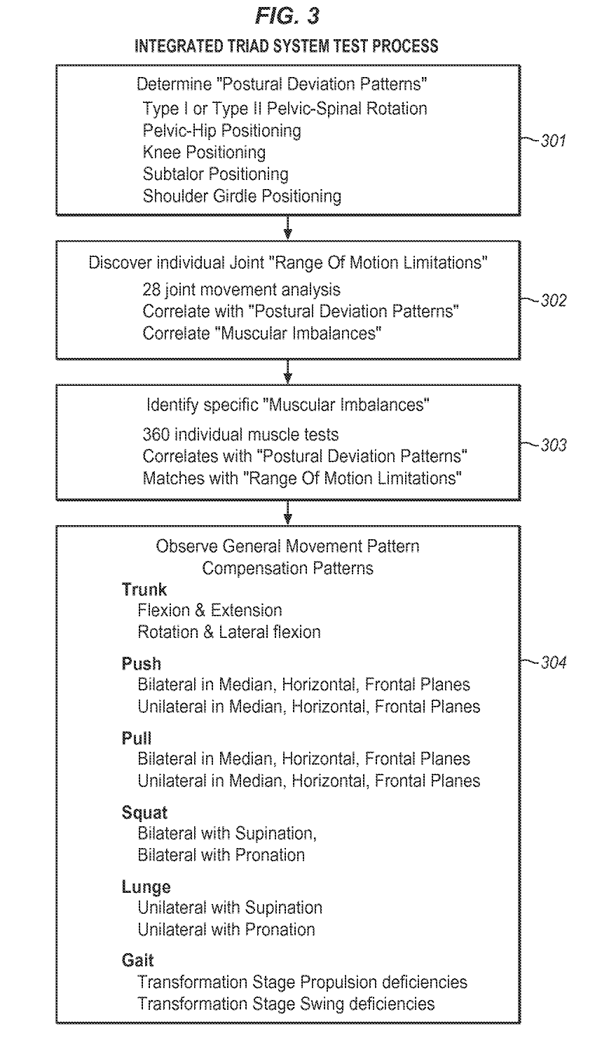

FIG. 1 illustrates, in block diagram form, the overall architecture of the present Ortho-Kinetics System 101, as embodied in the form of the Ortho-Kinetics CATT Application with at least one input device for receiving practitioner information and an output device for displaying the expert-derived information identified by the computer in response to a practitioner's query. FIG. 3 illustrates, in flow diagram form, the operation of the Ortho-Kinetics CATT Application 101. The present Ortho-Kinetics CATT Application 101 makes use of a database (in the form of either a computer-based system or tangible form of information) which stores expert-derived information to guide the practitioner in the performance of the following processes: advanced assessment methods 201 (termed “Ortho-Kinetics Assessment and Programming Processing” herein), corrective exercise and fitness training 202 (termed “Ortho-Kinetics Corrective and Performance Exercise Training” herein), along with specialized manual therapies 203 (termed “Ortho-Kinetics Integrated Therapy” herein). The data manipulation and computing process 112 that correlates the data input by the practitioner with the stored expert-derived analyses and exercise programs executes on server 114.

A certified practitioner who is equipped with at least one electronic device (such as an IPad, a computer 104 or 111, smart phone (not shown), interactive multi-media device (not shown), and the like (collectively termed “user equipment 104”)) makes use of the data input capabilities of the user equipment 104 to access the Ortho-Kinetics System 101 resident thereon or on an associated server 113. It should be noted that the Ortho-Kinetics CATT Application 101 may be resident on a remotely-located computer system 114, which can be accessed in well-known fashion by the certified practitioner using data communication capabilities of the user equipment 104. The architecture and implementation of the Ortho-Kinetics System 101 is described herein.

The Ortho-Kinetics System 101 can produce charts and queries to guide a practitioner in the use of the Ortho-Kinetics System 101 to identify specific postural deviations, specific muscular imbalances, treatment suggestions, and corrective exercise movements. In many cases, a visualization of the specific structures being tested and treated is beneficial to illustrate the deviations and imbalances discussed below to the client and to show the tests and treatments that are proposed.

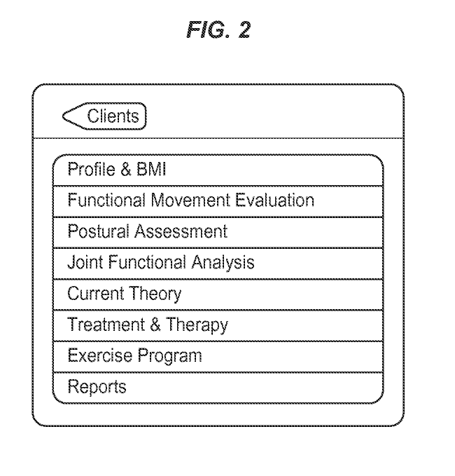

As shown in FIG. 2, the user is presented with a menu of options to access the various segments of the system as the testing, treatment, and training proceeds with a selected client. This menu also enables the practitioner to access previously input client data to enable the practitioner to update the data, review the comprehensive plan created for this client, and receive revised plans from the Ortho-Kinetics CATT Application 101. The operation of the various selections noted in this menu is described below.

### Specific Example of the Ortho-Kinetics CATT Application Operation

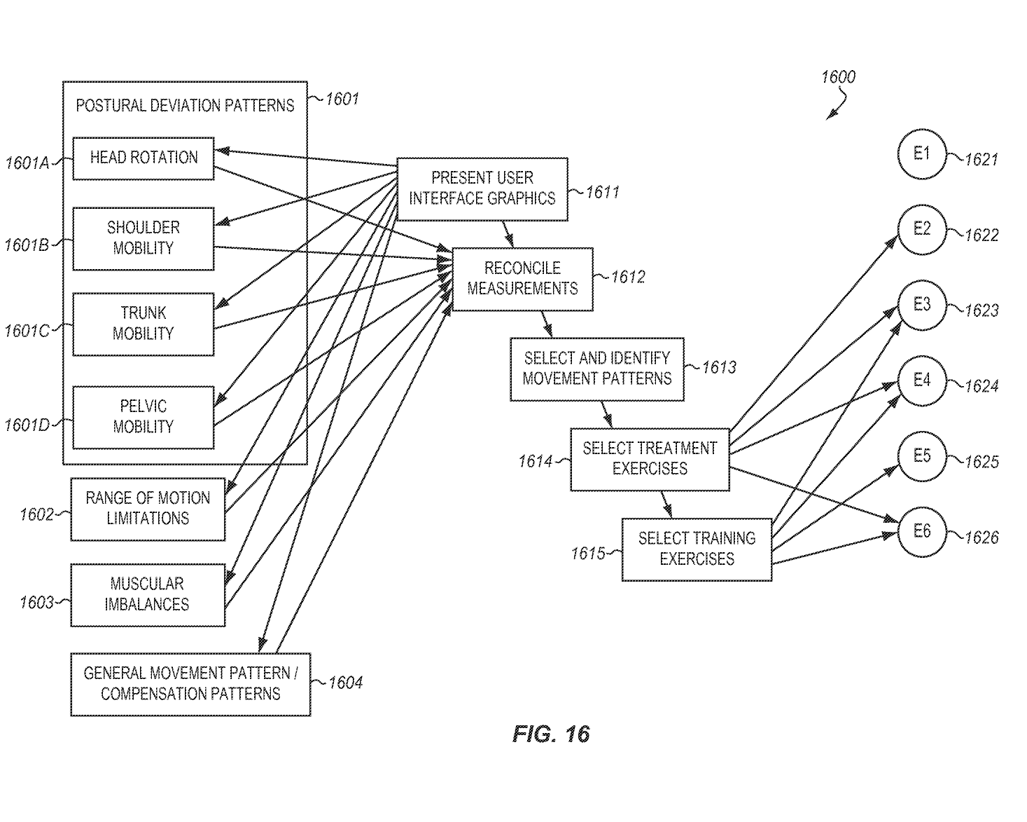

FIG. 16 illustrates the structure and operation of the expert system 1600 embodied in the Ortho-Kinetics CATT Application 101. In particular, as described herein, the Ortho-Kinetics CATT Application 101 implements a series of procedural steps to enable the practitioner to perform measurements on the client to identify problems associated with the deviations and imbalances present in the client. In particular, as described below, there are measurements taken of at least one of the following characteristics of the client's body: postural deviation patterns 1601, range of motion limitations 1602, muscular imbalances 1603, and general movement patterns/compensation patterns 1604. These are further subdivided into specific muscular/skeletal subsystems, as shown in part for the postural deviations 1601: head rotation 1601A, shoulder mobility 1601B, trunk mobility 1601C, and pelvic mobility 1601D. The practitioner is provided with displays of each of these subsystems by the Ortho-Kinetics CATT Application 101 at process 1611, and the practitioner is enabled to take measurements on the client and input data illustrative of these measurements in process 1612.

The received data is processed by the expert system 1600 in process 1613 to select and identify a set of movement patterns which are key to resolving the client's muscular/skeletal problems. The expert system 1600 in process 1614 translates the identified movement patterns into a selection of treatment exercises from the vast library of such exercises that are stored in the memory of the Ortho-Kinetics CATT Application 101. These exercises are displayed in part by exercises E1-E6 (items 1621-1626 on FIG. 16), which exercises are selected (E2-E4, E6) to address the problems identified by the measurements taken by the practitioner. Furthermore, the expert system 1600 also executes process 1615, which identifies a selection of training exercises (E3-E6) which the client can use to educate their body to create muscle memory to correct their imbalances.

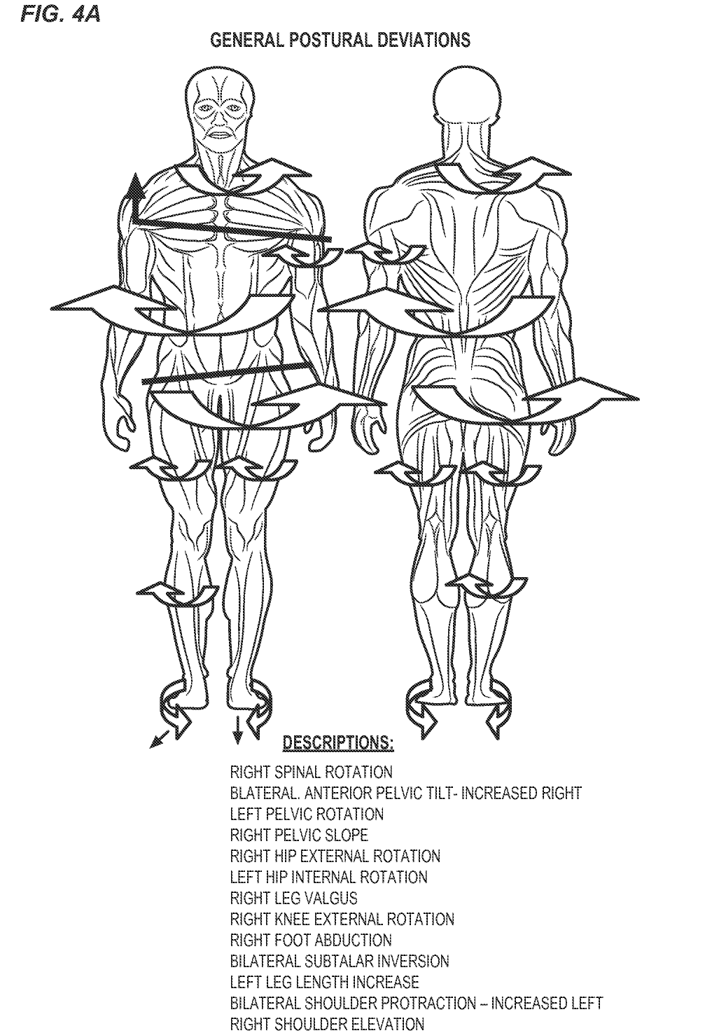

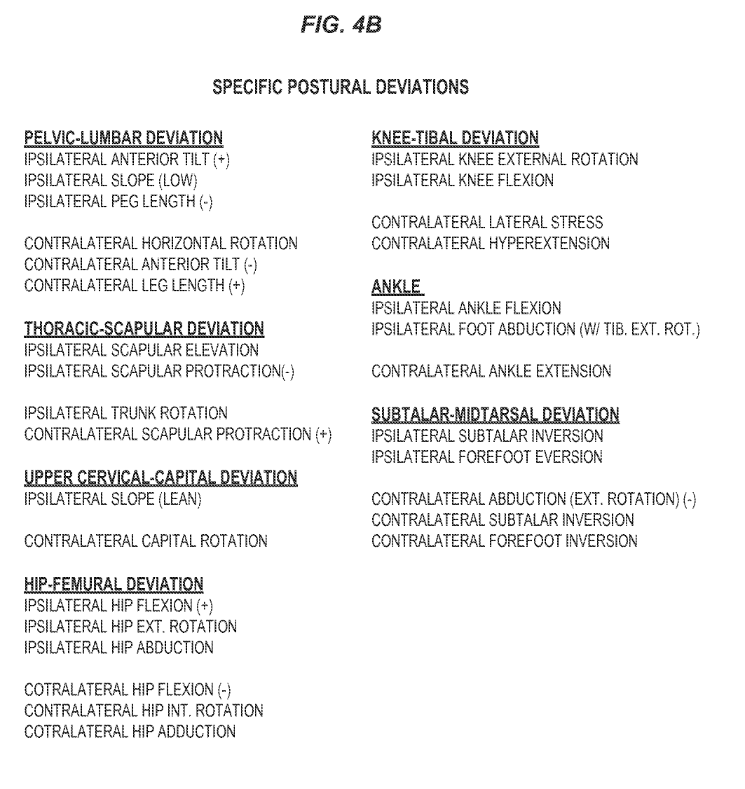

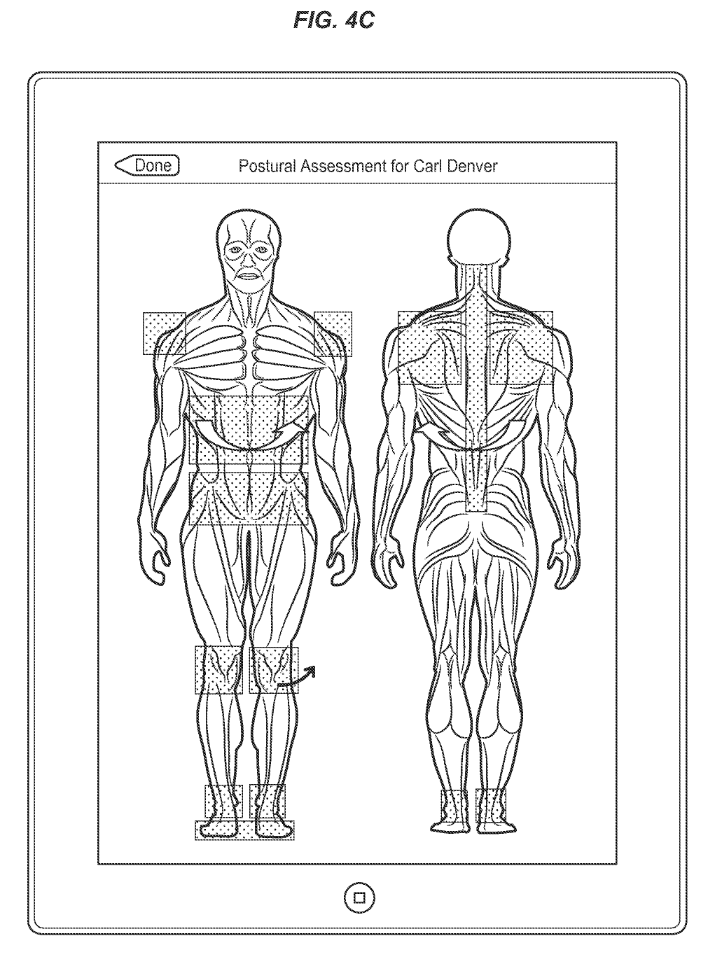

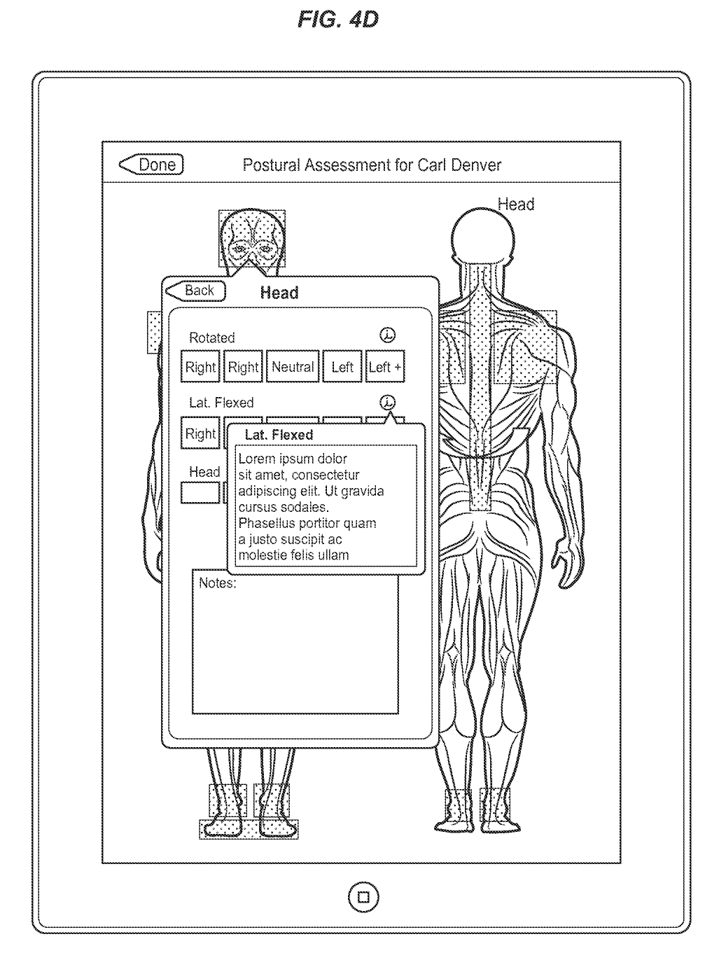

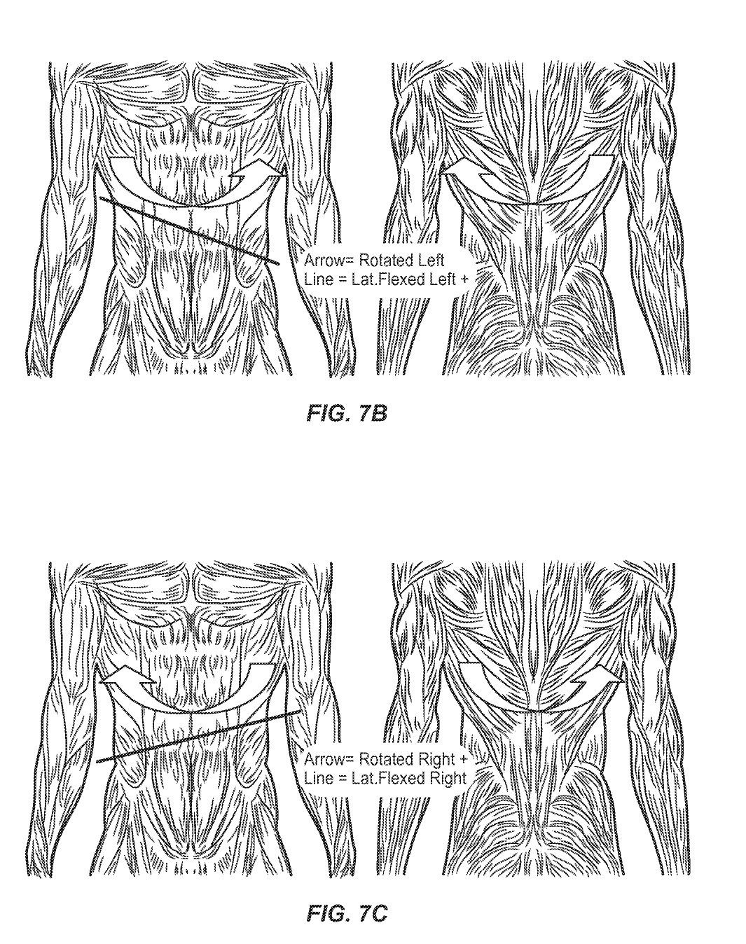

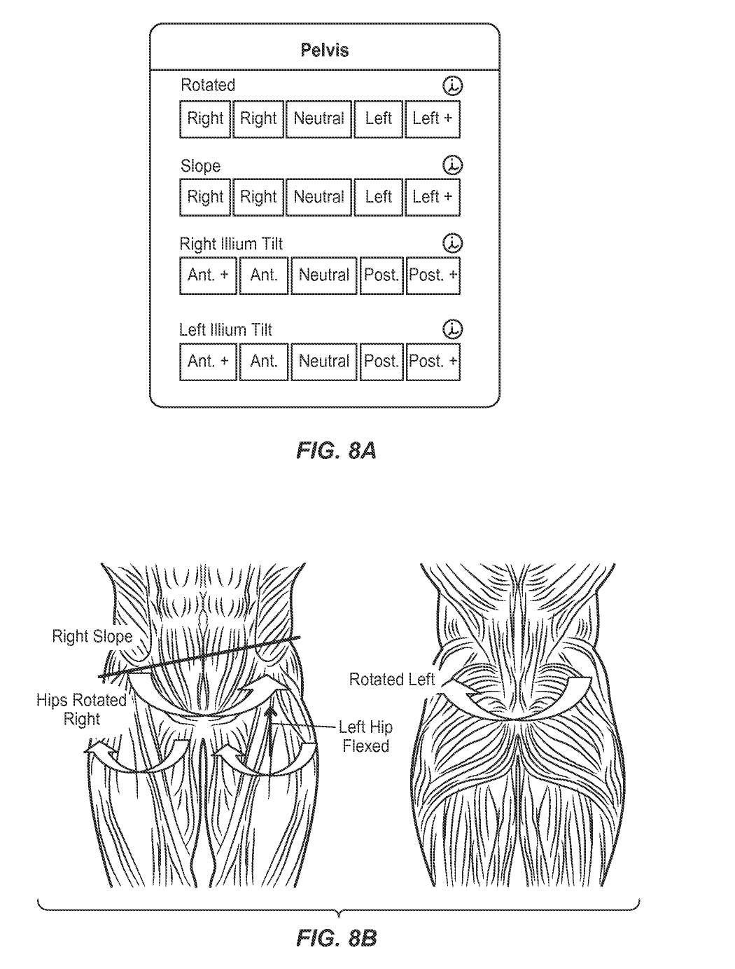

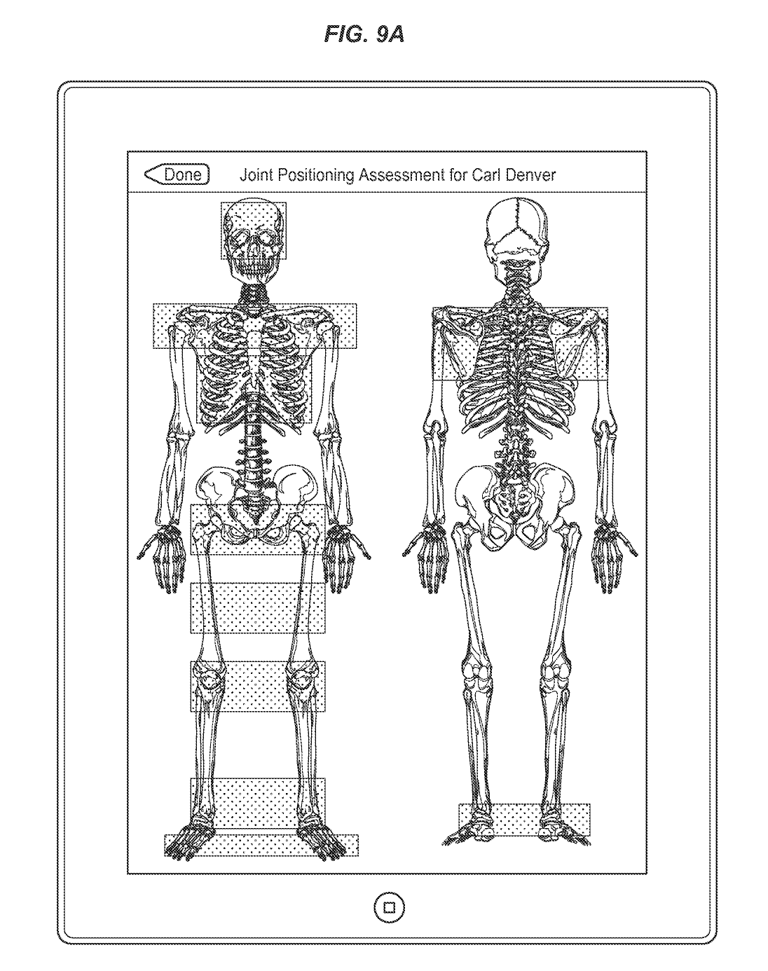

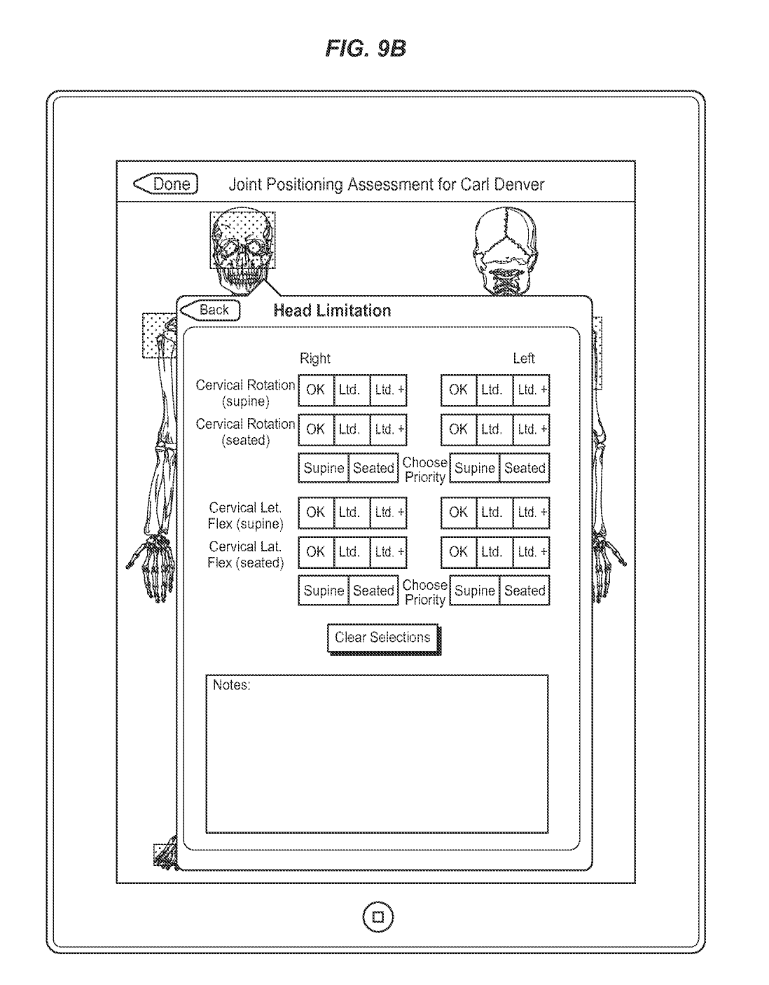

FIGS. 4A and 4B illustrate an example of charts produced by the Ortho-Kinetics CATT Application 101 to guide a practitioner in the use of the Ortho-Kinetics CATT Application 101 in identifying specific postural deviations; FIGS. 7A-7C illustrate an example of charts produced by the Ortho-Kinetics CATT Application 101 to guide a practitioner in the use of the Ortho-Kinetics CATT Application 101 in identifying specific muscular imbalances; FIG. 8 illustrates an example of a chart produced by the Ortho-Kinetics CATT Application 101 to guide a practitioner in the use of the Ortho-Kinetics CATT Application 101 in identifying treatment suggestions; and FIG. 9 illustrates an example of a chart produced by the Ortho-Kinetics CATT Application 101 to guide a practitioner in the use of the Ortho-Kinetics CATT Application 101 in identifying corrective exercise movements.

These charts are illustrative of the process implemented by the Ortho-Kinetics CATT Application 101 and are but a few examples of a portion of the human body that can be treated using the Ortho-Kinetics CATT Application 101.

### The Ortho-Kinetics CATT Application Assessment and Programming Process

FIG. 3 illustrates, in flow diagram form, the operation of the Ortho-Kinetics CATT Application Assessment and Programming Process. The Ortho-Kinetics CATT Application Assessment Process is an automated computer process that makes use of a biomechanically-based system of combined Postural Pattern Assessment, Functional Movement Evaluation, and detailed Joint Bias Analysis. Together these advanced assessment methods accurately identify postural deviations, discover muscular imbalances and joint motion bias, and unveil functional movement compensations that collectively create further neuromuscular imbalances, continued or progressed postural deviations, or reduced joint function, and can cause pain, inhibit performance, and deteriorate overall health.

This comprehensive assessment begins with an in-depth interview of the client by the practitioner that is designed to establish fitness objectives, set realistic performance goals, and determine any specials needs or challenges that may interfere with obtaining optimal results. The process then includes the operation of an automated computer process that guides the trainer or therapist through a method of postural evaluation, followed by a detailed joint-by-joint stability and range-of-motion analysis. This detailed information then is correlated with a Functional Movement Evaluation and Cardiovascular Readiness test that collectively helps to determine the best exercise program and treatment plan to begin for progressing towards addressing a client's needs and accomplishing their goals.

As shown in FIG. 3, the Ortho-Kinetics CATT Application Assessment Process 300 represents an in-depth series of tests to gather adequate data regarding the evaluation of the client. At step 301, the practitioner determines “Postural Deviation Patterns,” which include, but are not limited to: Type I or Type II Pelvic-Spinal Rotation; Pelvic-Hip Positioning; Knee Positioning; Subtalor Positioning; and Shoulder Girdle Positioning, as shown in FIGS. 4A and 4B. FIG. 4A illustrates an example of a chart produced by the Ortho-Kinetics CATT Application to guide a practitioner in the use of the Ortho-Kinetics CATT Application in identifying specific postural deviations. FIG. 4C illustrates the screen produced by the Ortho-Kinetics CATT Application 101 to enable a practitioner to create a new postural assessment, update an existing one, or complete an unfinished assessment. Due to the number of interactions possible on this screen, this description contains general rules in addition to specific sections on each assessment action that can be taken. A practitioner can touch or click on any square to log the assessment results for that area. That action will bring up an assessment popup as shown in the screenshot in FIG. 4D. Touching the information icon to the left of the assessment item will bring up a popup containing instructions. As the practitioner selects the appropriate answer for the assessment item, the appropriate line or arrow is drawn on the front or back view of the muscle man (as shown on his left knee in the screen shot in FIG. 4C).

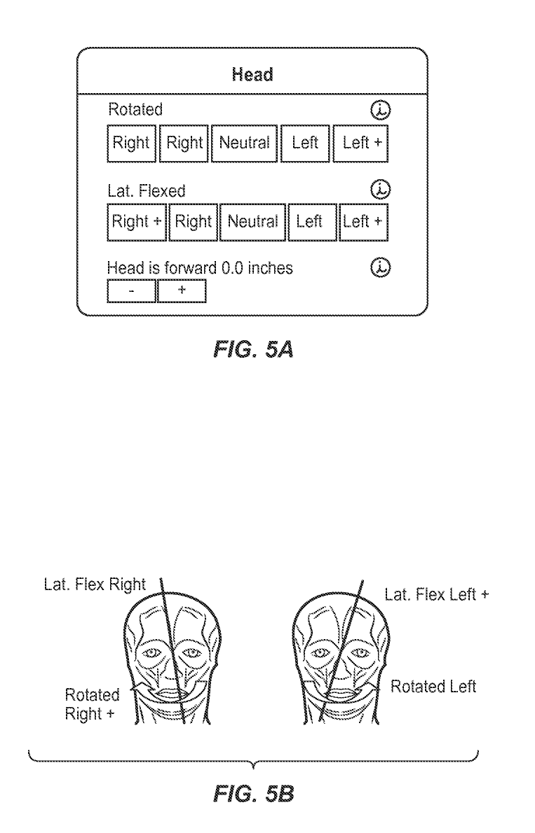

For example, the assessment screens for Head Rotation are illustrated in FIGS. 5A and 5B, and present the front view and the rear view (not shown) of the head. The direction of the arrow on the rear view is opposite of the arrow on the front view. The maximum forward head position is 5.0 inches. The plus and minus buttons increment the total inches by ½ inch. The practitioner determines the ability of the client to rotate and flex their head and inputs this data via the screen display of FIG. 5A.

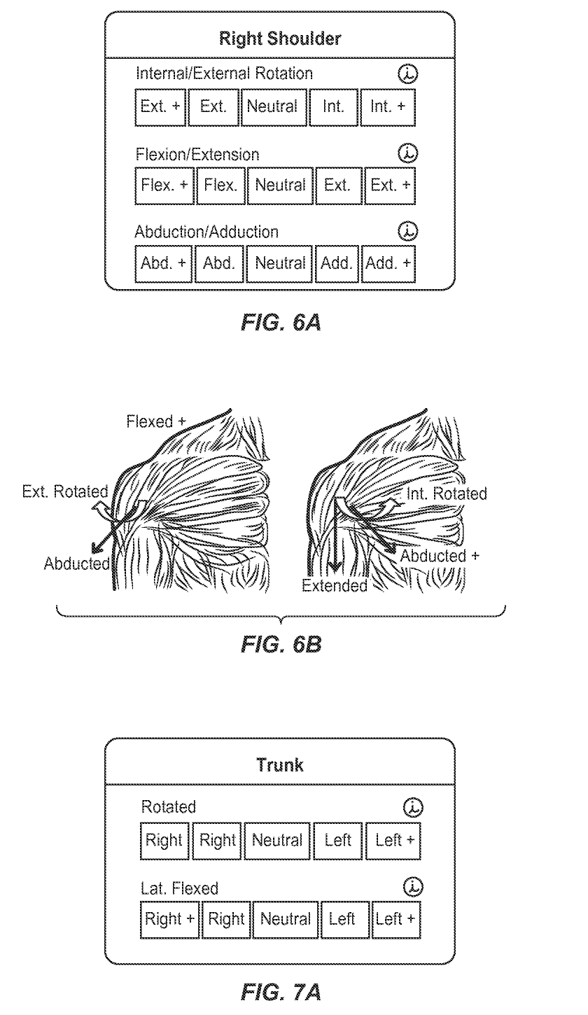

Similarly, the assessment screens for the shoulder, trunk, and pelvis are illustrated in FIGS. 6A and 6B, 7A-7C, and 8A and 8B, respectively. Pelvic rotation measurements provide an indicator for hip rotation. Therefore, if the pelvis is rotated to the right, the left hip is externally rotated and the right hip is internally rotated and the appropriate arrows are drawn on the hips. If the pelvis is rotated to the left, the left hip is internally rotated and the right hip is externally rotated and the appropriate arrows are drawn on the hips. No lines or arrows are drawn for anterior/posterior tilt of the ilium; however, the positioning of the ilium allows the Ortho-Kinetics CATT Application to make assumptions about the positioning of the actual hip joint that will be confirmed during the Joint Positioning Assessment. The following rules apply for each individual ilium-hip pair (e.g., Right Ilium and Right Hip):
(a) If the ilium has an anterior tilt, the hip will be flexed and the appropriate arrow will be drawn on the hip;
(b) If the ilium has a posterior tilt, the hip will be extended and the appropriate arrow will be drawn on the hip; and
(c) Extreme tilt will result in extreme flexion/extension.

Similar to the ilium-hip relationship, the Ortho-Kinetics CATT Application 101 infers the abduction/adduction of the individual hip joints based on the pelvic slope. If the pelvis drops or slopes to the right, the right hip is abducted and the left hip is adducted. No arrows or lines are drawn for this. If the pelvis drops or slopes to the left, the right hip is adducted and the left hip is abducted. No arrows or lines are drawn for this. Extreme slope or drop results in extreme adduction/abduction. No arrows or lines are drawn for this.

The practitioner also can proceed with a joint analysis for the Knee, Ankle, Midtarsal, Spine, Scapula, and Subtolar to provide data input to the Ortho-Kinetics CATT Application 101 on these subsystems. These processes are analogous to those just described and, for the purposes of brevity, are not presented in detail herein. Suffice it to say, the Ortho-Kinetics CATT Application 101 automatically steps the practitioner through data gathering steps on the noted segments of the client's body to perform a complete postural analysis of the client.

At step 302, the practitioner then proceeds to test the client to discover individual Joint “Range Of Motion Limitations,” which include, but are not limited to: 28-joint movement analysis; correlate with “Postural Deviation Patterns”; and correlate with “Muscular Imbalances,” as shown in FIGS. 9A and 9B. The primary difference between the Postural Assessment and the Joint Analysis is that the Joint Analysis is looking for where the joint cannot go' whereas the Postural Assessment is looking for where the joint naturally goes. This is also called a “joint bias.” While a joint naturally can only go in one direction, it is possible for a joint to be limited in both directions. This makes comparing the two analyses to each other difficult because the answer scale is different. The logic below explains how to map the two joint limitation responses into a joint preference response for easy comparison to the Postural Assessment. If a joint is limited (or limited+) in both directions, then the joint is considered “immobile” except for the head, trunk, pelvis, and spine. The range of motion measurements include:

Limited in flexion/extension

Limited in adduction/abduction

Limited in rotation to right and left

Limited in lateral flexion to right and left

Limited in external and internal rotation

Limited in elevation and depression

Limited in protraction and retraction

Limited in eversion and inversion

The above rule is true for all joints, but it is not true for the spine-related areas: head, trunk, pelvis, and spine. For these areas, the immobile symbol is only drawn if it is immobile to the same degree in both directions. For example, if the head is limited in rotation to the left and to the right, the immobile symbol will be drawn. But, if the head is limited in rotation to the left and limited+in rotation to the right, the arrow will be drawn for the limited+rotation to the right. If a joint is limited in one direction, then the Joint Preference is the opposite of the limitation; e.g., if a right shoulder is limited in external rotation and okay in internal rotation, then the Joint Preference would be Internal Rotation. Also, if a left shoulder is limited+in external rotation and okay in internal rotation, then the Joint Preference would be Internal Rotation+. If a joint is not limited in either direction (practitioner chooses for both directions), then the Joint Preference would be Neutral.

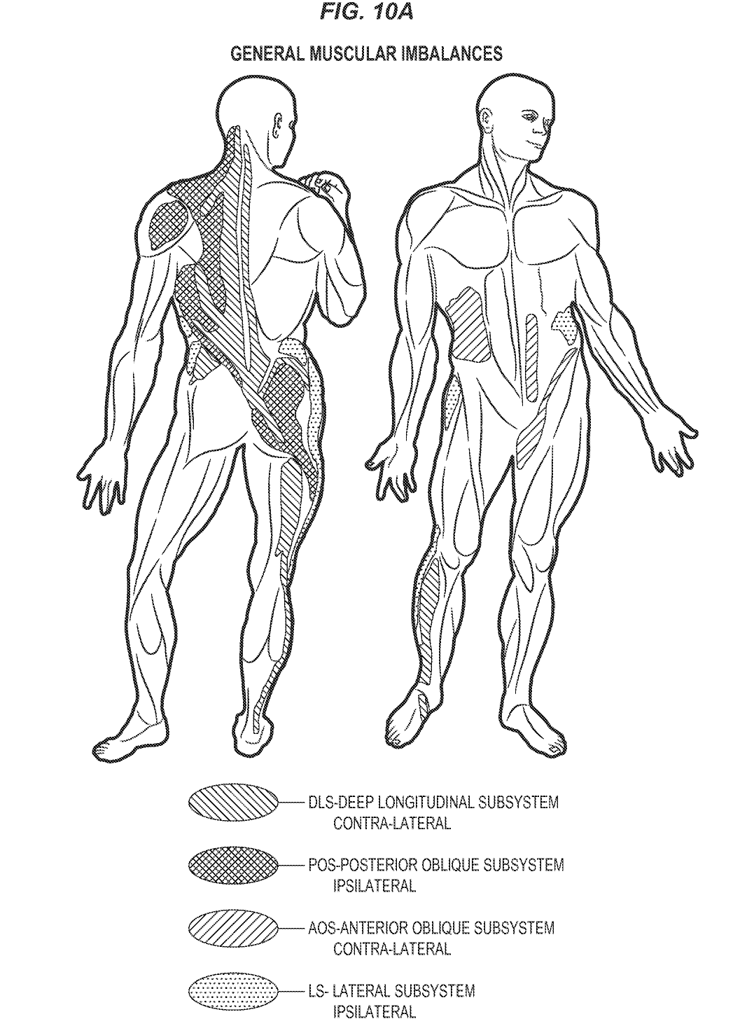

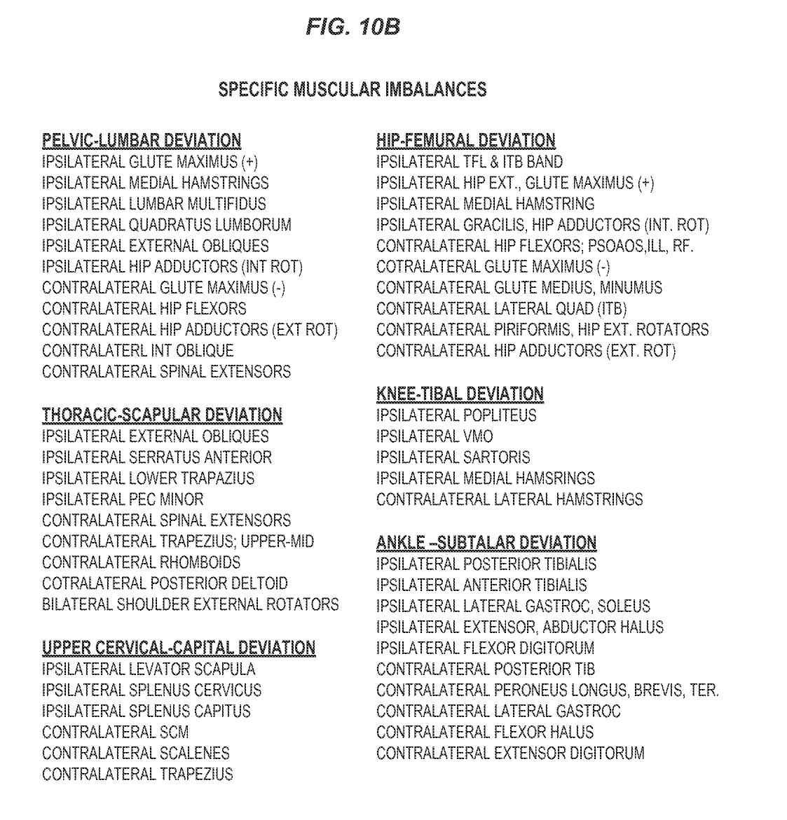

At step 303, the practitioner identifies specific “Muscular Imbalances,” which include, but are not limited to: 360 individual muscle tests; correlation with “Postural Deviation Patterns”; and matching of “Range Of Motion Limitations,” as shown in FIGS. 10A and 10B, which illustrate an example of a chart produced by the Ortho-Kinetics CATT Application 101 to guide a practitioner in the use of the Ortho-Kinetics CATT Application 101 in identifying specific muscular imbalances. The Ortho-Kinetics CATT Application 101 query screens and data input processes are analogous to those described above and again are not described in detail for the sake of brevity.

Finally, at step 304, the practitioner evaluates the Functional Movement Pattern Compensation which includes, but is not limited to: Trunk (including Flexion and Extension, and Rotation and Lateral Flexion); Push (Bilateral In Median, Horizontal, and Frontal Planes; and Unilateral in Median, Horizontal, and Frontal Planes); Pull (Bilateral in Median, Horizontal, and Frontal Planes; and Unilateral in Median, Horizontal, and Frontal Planes); Squat (Bilateral with Supination; and Bilateral with Pronation); Lunge (Unilateral with Supination; and Unilateral with Pronation) and also Gait (Transformation Stage Propulsion deficiencies; and Transformation Stage Swing deficiencies). The Ortho-Kinetics CATT Application 101 query screens and data input processes are analogous to those described above and again are not described in detail for the sake of brevity.

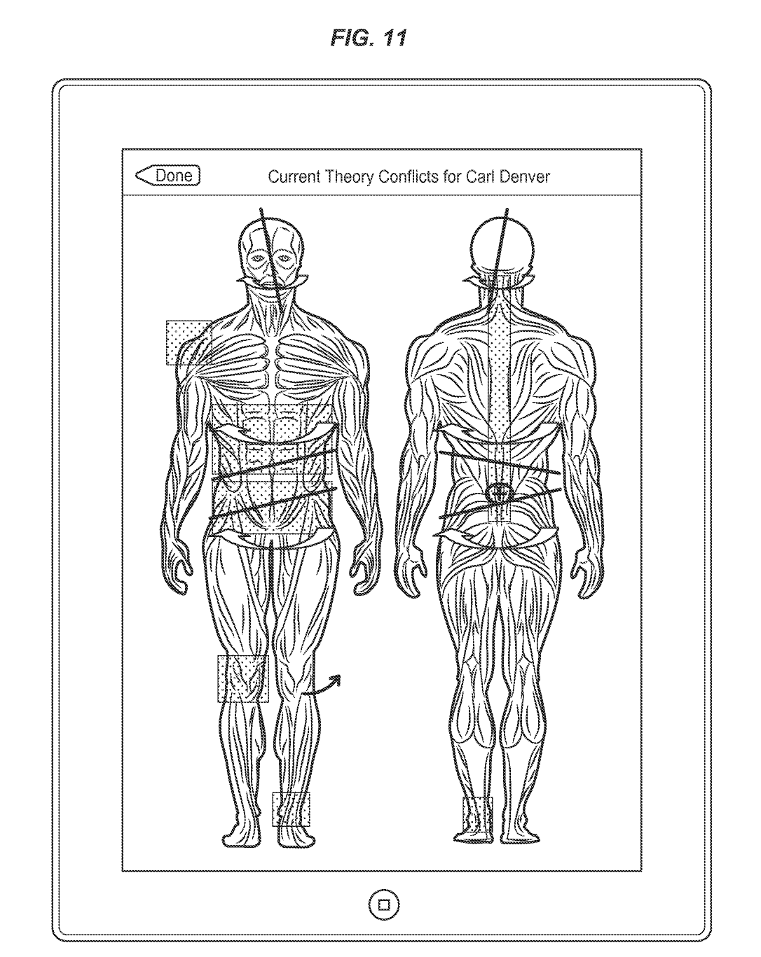

The Ortho-Kinetics CATT Application 101 produces an intelligent summary of what was found on the Postural Assessment and the Joint Analysis. In the likely scenario that the two assessments do not agree on a specific test, the practitioner must pick which assessment should be used for the creation of a treatment procedure. Once the conflicts are determined, the image of FIG. 11 is displayed to the practitioner. The arrows/lines for the non-conflicting items are drawn on the figure and boxes appear for the items where conflicts exist. When the practitioner touches one of the boxes, a Conflict Resolution Popup appears and the practitioner can input data to resolve the conflict.

This collection of tests and analyses enable the practitioner, using the Ortho-Kinetics CATT Application 101, to produce an accurate picture of the client's postural and muscular deviations.

### The Ortho-Kinetics CATT Application Treatment Process

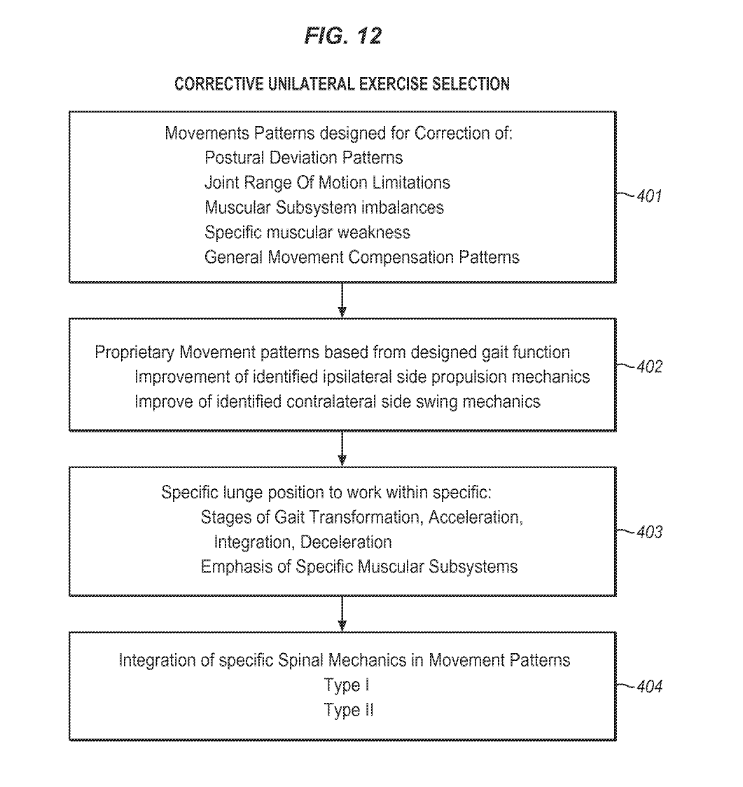

FIG. 12 illustrates, in flow diagram form, the operation of the Ortho-Kinetics CATT Application Treatment Process 400. The Ortho-Kinetics CATT Application Treatment Process 400 entails the use of a combination of specialized manual therapies such as Active Release Techniques® and Muscle Activation Techniques, as well as specific corrective exercises to increase joint function, create stability, and improve flexibility.

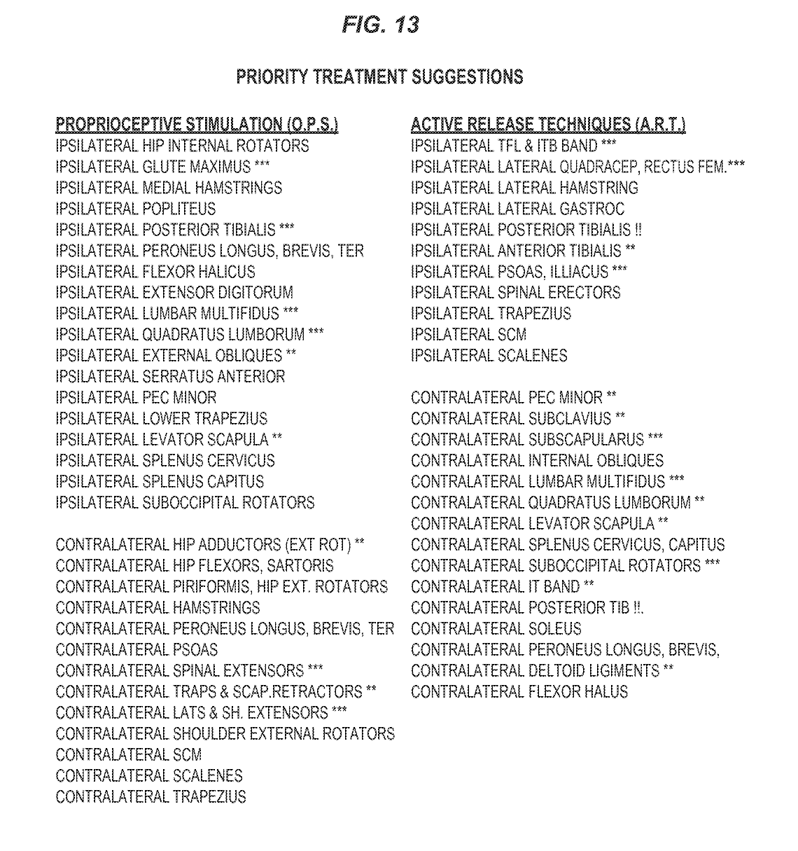

Based upon the data collected in the Ortho-Kinetics CATT Application Test Process 300, the practitioner can proceed with the next step in the process, which is the Ortho-Kinetics System Treatment Process 400. As shown in FIGS. 12 and 13, the Ortho-Kinetics CATT Application 101 maps the data collected in the Ortho-Kinetics System Test Process 300 into a set of Priority Treatment Suggestions to implement the Ortho-Kinetics CATT Application Treatment Process 400.

At step 401, the practitioner reviews the set of Movement Patterns designed for correction of: Postural Deviation Patterns; Joint Range-Of-Motion Limitations; Muscular Subsystem Imbalances; Specific Muscular Weakness; and General Movement Compensation Patterns. Specific examples of these suggestions are shown in FIG. 8. Thus, the Ortho-Kinetics CATT Application Treatment Process 400 is responsive to the data input by the practitioner for this client to process the data and produce client-specific recommendations which cover all of the areas which require treatment. Since many of these areas are interrelated, it is typically difficult for a practitioner to identify every anomaly and the linkages among the various symptoms which evidence each problem. Thus, at step 402 in this example, the practitioner receives data indicative of Movement patterns based from designed gait function, which include, but are not limited to: improvement of identified ipsilateral side propulsion mechanics and improvement of identified contralateral side swing mechanics. Similarly, at step 403, the practitioner receives data indicative of specific lunge positions to work within specific: Stages of Gait Transformation, Acceleration, Integration, Deceleration, and Emphasis of Specific Muscular Subsystems. Finally, at step 404, the Ortho-Kinetics CATT Application Treatment Process 400 identifies the integration of specific Spinal Mechanics in Movement Patterns, including Type I and Type II Postural Patterns.

### The Ortho-Kinetics CATT Application Training Process

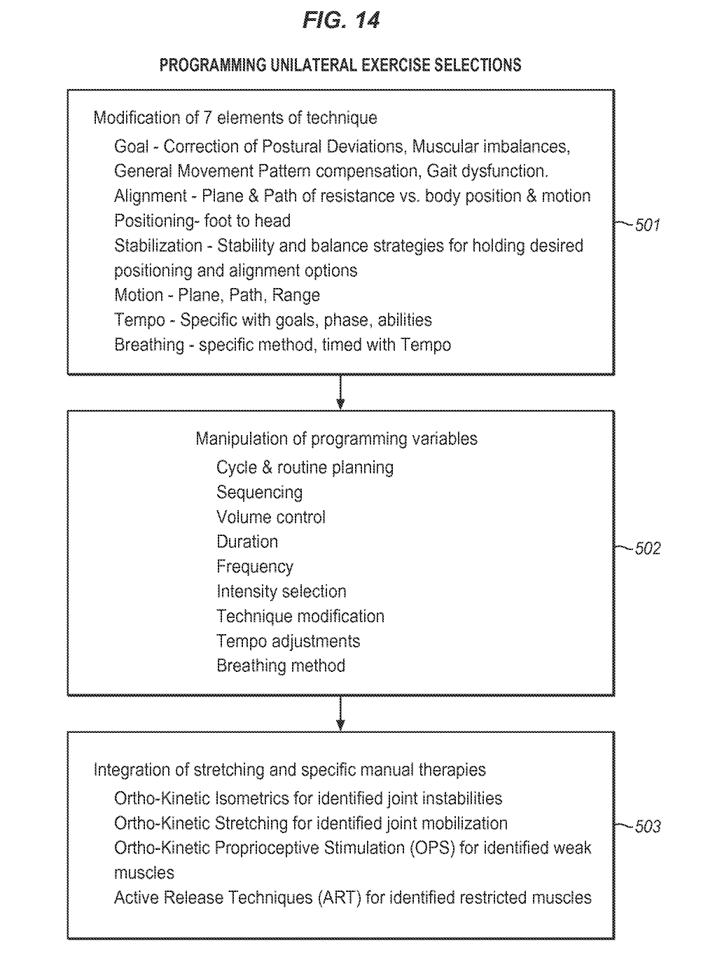

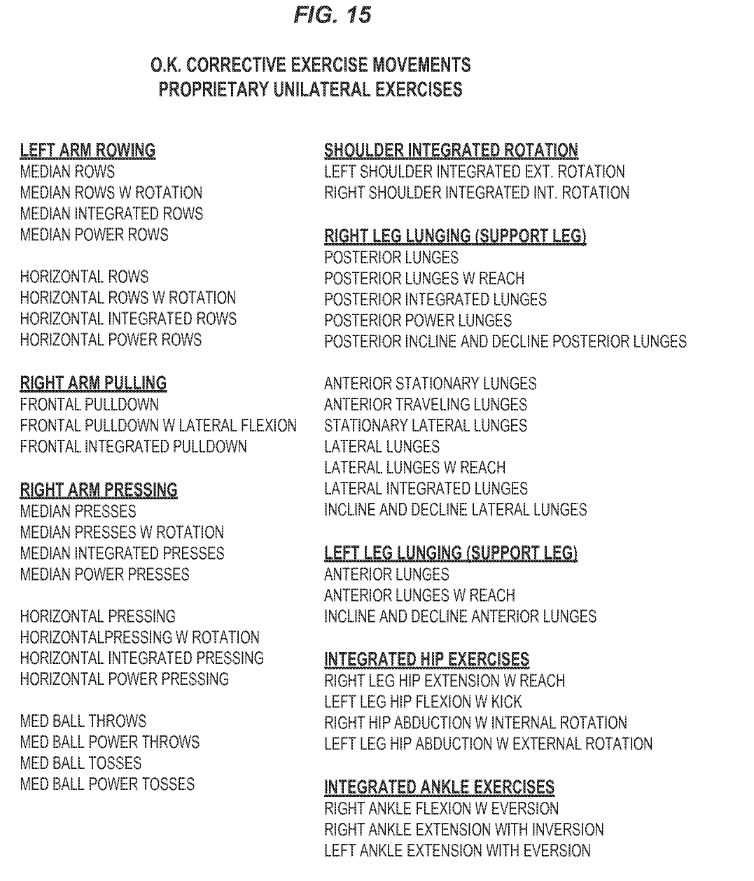

FIG. 14 illustrates, in flow diagram form, the operation of the Ortho-Kinetics CATT Application Training Process 500. Based directly from the information discovered through the comprehensive assessment process, specific corrective exercise movements are selected to help realign posture, correct muscular imbalances, and improve movement ability. These functional and performance-based exercises can be modified, sequenced, and combined with more goal-centered exercises and modalities to create weekly routines and long-term programs that meet a client's needs and accomplish a client's objectives.

Each Training exercise is designed to create muscle memory to enable the client to replicate the proper movement or sequence of movements as a result of their body and mind being conditioned to move in the proper mode. Each of the Training exercises illustrated identifies the name of the exercise and identifies key information: identification of the primary muscles which are being trained, identification of the subsystem and core portion of the body, and identification in a step-wise fashion of the proper performance of the Training exercise. Graphical illustrations may also be provided to assist the client in the visualizing of the Training exercise. These materials may be reproduced in whole or in part in tangible form to assist the client and Practitioner to operate as a team in the implementation of the Training exercise. The Practitioner monitors the client's implementation of the Training exercise and provides corrective instruction to the client to ensure the Training exercise is being precisely performed. FIG. 9 illustrates an example of a chart produced by the Ortho-Kinetics CATT Application 101 to guide a practitioner in the use of the Ortho-Kinetics CATT Application 101 in identifying corrective exercise movements.

The Ortho-Kinetics System Corrective and Performance Exercise Training Process 500 next presents the practitioner with data indicative of customized Programming Unilateral Exercise Selections which are selected specifically for this client. At step 501, the Ortho-Kinetics Corrective and Performance Exercise Training Process 500 presents data relevant to the modification of seven elements of technique which are used by the practitioner. The Ortho-Kinetics Corrective and Performance Exercise Training Process 500 presents the following data representative of the Goals: Correction of Postural Deviations, Muscular Imbalances, Functional Movement Pattern Compensation, and Gait Dysfunction. These goals are achieved by the creation of an exercise plan which addresses the following aspects: Alignment—Plane and Path of Resistance vs. Body Position and Motion; Positioning—Foot to Head; Stabilization—Stability and Balance Strategies for Holding Desired Positioning and Alignment Options; Motion—Plane, Path, and Range; Tempo—Specific with Goals, Phase, and Abilities; and Breathing—Specific Method Timed with Tempo. Finally, at step 502, the Ortho-Kinetics Corrective and Performance Exercise Training Process 500 defines an entire ongoing process which addresses the manipulation of programming variables. These include, but are not limited to: Cycle and Routine Planning; Sequencing; Volume Control; Duration; Frequency; Intensity Selection; Technique Modification; and Tempo Adjustments. Also included in this are Breathing Methods.

In order to properly address these exercises using the most appropriate techniques, at step 503, the Ortho-Kinetics Corrective and Performance Exercise Training Process 500 provides an integration of stretching and specific manual therapies including, but not limited to, the existing processes: Ortho-Kinetic Isometrics for identified joint instabilities; Ortho-Kinetic Stretching for identified joint mobilization; Ortho-Kinetic Proprioceptive Stimulation (OPS) for identified weak muscles; and Active Release Techniques® (ART) for identified restricted muscles.

### The Ortho-Kinetics CATT Application Unilateral Exercises

Features:
(a) The specific increased functional demands of targeted muscles and subsystems for improved intra-muscular and inter-muscular recruitment sequencing;
(b) The specific lengthening/stretching of antagonist muscles and subsystems for improved joint movement ability;
(c) The specific neurological re-programming of muscles for the development of more efficient motor control and enhanced coordination of integrated joint movement patterns; and
(d) Improved muscular recruitment sequencing, increased joint movement ability, neurological re-programming, and enhanced motor control which are all critical for correcting muscular imbalances and eliminating movement pattern compensation.

Only through effective selection, programming, and implementation of specific exercise movements performed with specific techniques can these benefits be potentially derived from a fitness program.

Random exercise selection, non-specific or faulty exercise technique, and totally subjective or aesthetic-based programming can only result in random adaptation, faulty motor programming, and reinforcement of compensation.

Selection of specific Ortho-Kinetics Corrective and Performance Exercise Process 500 corrective exercises, particularly the unilateral exercises, are determined from the information gathered from the Ortho-Kinetics CATT Application Test Process 300 and related to correcting postural deviations, muscular imbalances, range-of-motion deficits, and movement compensations.

### Specifics of Ortho-Kinetics CATT Application Unilateral Exercise Technique

The use of specific lunge positions for each exercise are modified for each person's present abilities based on research of functional movement patterns and efficient gait performance:
(a) Specific positioning affects alignment of force application and recruitment of specific muscles and subsystems; and
(b) Neural re-programming and motor control encompass joint stabilization strategies, as well as joint motion, which are dictated in part by positioning options.

The intentional integration and specific use of Type I and Type II spinal motion in each exercise movement pattern prescribes:
(a) Specific synchronized tri-plane movement control of the entire spine will dictate intra-muscular and inter-muscular recruitment sequencing and neural programming; and
(b) Selection and modification of specific exercises and techniques containing specific Type I and Type II movement patterns are based from objective results of individual assessment, not random or subjective goals.

Progression of specific Ortho-Kinetics Corrective and Performance Exercise 101 selection and technique are:
(a) Unilateral Push and Pull and Lunge: Working from Type I rotation to neutral;
(b) Unilateral Push and Pull with Rotation and Lunge with Reach: Working from Type I rotation to contralateral Type I rotation;
(c) Unilateral Integrated Push and Pull and Integrated Lunge: Working from Type I rotation to Type I contralateral rotation with pelvic plane change;
(d) Unilateral Power Push and Pull and Plyo Lunges: Working from Type I rotation to Type I contralateral rotation with pelvic plane change and dynamic lunging;
(e) Integrated Medicine Ball Throws and Tosses: Working from Type I or Type II rotation to contralateral Type I rotation with pelvic plane change and dynamic lunging with medicine ball throws and tosses; and
(f) Plyometrics and Agility: Working from Type I or Type II rotation to Type I contralateral rotation with pelvic plane change and dynamic lunging with Propulsion or Swing Emphasis.

### The Integration and Application of Ortho-Kinetics System Active Stretching and Isometrics

The Ortho-Kinetics System stretching and isometrics as a part of the Ortho-Kinetics Corrective and Performance Exercise Process 500 is based on the concepts that the neuromuscular system has a balance of length/tension ratios for each muscle that is “optimal” for the specific person in order for them to perform at peak performance.

“Normal” ratios of length/tension are a learned normal based on physiological adaptations, so they vary from person to person and from muscle to muscle with “normal” not meaning optimal and typically involving compensation. Therefore, the lengthening of a muscle is only as important as its ability to also produce adequate force.

Joint mobility is only as important as joint stability. As changes in the “flexibility” or strength levels of a muscle changes, certain reciprocal changes within the muscle must also change. As changes in the “flexibility” or strength levels of a muscle changes, certain associated changes in antagonistic and synergistic muscles also will occur. Therefore, stretching, particularly of a passive type, without integration of intra-muscular and inter-muscular strengthening is unlikely to be effective and can even be detrimental to overall performance.

The Ortho-Kinetics System stretching will only provide active stretching techniques designed to simultaneously improve muscle strength along with flexibility and develop joint stability as well as joint mobility. Selection of specific Ortho-Kinetics System stretching exercises will be determined from the Ortho-Kinetics CATT Application Test Process 300 and related to correcting postural deviations, muscular imbalances, range-of-motion deficits, and movement compensations.

### The Integration And Application Of Specific Manual Therapies

There are existing manual therapies that can be used in this process, such as Ortho-Kinetic Proprioceptive Stimulation (OPS) and Active Release Techniques® (ART). The integration of manual therapies and soft tissue treatment techniques as a part of the Ortho-Kinetics process is based on the concept that the neuromuscular system has a balance of length/tension ratios for each muscle that is “optimal” for the specific person in order for them to perform at peak performance.

Development of “ideal” ratios of length/tension for the neuromuscular system is a process, as it requires new physiological adaptations as well as neurological reprogramming to improve biomechanical function for peak performance. Therefore, the lengthening or strengthening of a muscle is only as important as its ability to also provide the complimentary ability to lengthen or produce adequate force. Joint mobility is only as important as joint stability.

As changes in the “flexibility” or strength levels of a muscle changes, certain reciprocal changes within the muscle must also change. As changes in the “flexibility” or strength levels of a muscle changes, certain associated changes in antagonistic and synergistic muscles also will occur. Therefore, integration of soft tissue techniques designed to stimulate proprioception for improved muscular force production without also maintaining the optimal lengthening ability of the muscle, synergistic muscles, or antagonist muscles, will most likely be ineffective and unlikely to hold its newly acquired level of improvements. Concurrently, integration of techniques designed to improve muscle lengthening ability and increase joint range of motion (such as Active Release Techniques®), without also improving or maintaining the optimal strengthening of the muscle and/or associated synergist or antagonist muscles, also will be less likely to hold its newly acquired level of improvements. Selection and integration of specific soft tissue techniques will be determined from the Ortho-Kinetics System Assessment and Programming Process and related to correcting postural deviations, muscular imbalances, range-of-motion deficits, and movement compensations.

### Summary

The Ortho-Kinetics System implements a comprehensive system of combining advanced assessment methods, corrective exercise, and fitness training, along with specialized manual therapies designed to develop optimal human performance, using either computer-based or conventional tangible media technology to guide the practitioner.

What is claimed as new and desired to be protected by Letters Patent of the United States is:

1. A method for testing and treating a person to improve their movement ability, the method comprising the steps of:
automatically presenting, from a computer system to a practitioner, a sequence of computer generated images to guide the practitioner to perform postural pattern assessments, functional movement evaluation, and detailed joint motion analysis on the person;
inputting, into the computer system by the practitioner, measurement data taken on the person by the practitioner in executing the postural assessment, functional movement pattern evaluation, and detailed joint motion analysis on the person;
automatically identifying postural deviations, discovering neuromuscular imbalances, and unveiling movement limitations by the computer processing the data input by the practitioner; and
automatically generating, by the computer system using a computerized database system and based upon the step of automatically identifying, a set of specific corrective and performance exercises along with specialized manual therapies to be used to design overall exercise programs and treatment plans for the person to perform to increase their joint function, create stability, and improve flexibility.

2. The method of testing and treating a person of claim 1 wherein said step of inputting comprises:
conducting an in-depth interview of the person designed to establish fitness objectives, set realistic performance goals, and determine any specials needs or challenges that may interfere with obtaining optimal results.

3. The method of testing and treating a person of claim 2 wherein said step of inputting further comprises:
executing a postural pattern assessment of the person by a practitioner, followed by a detailed joint-by-joint stability and range-of-motion analysis of the person.

4. The method of testing and treating a person of claim 3 wherein said step of automatically generating further comprises:
correlating detailed information gathered in the step of inputting with a Functional Movement Pattern Evaluation and General Exercise Readiness Testing to determine the best exercise program and treatment plan for the person.

5. The method of testing and treating a person of claim 1 wherein said step of automatically presenting comprises:
implementing a biomechanically-based system of combined postural pattern assessment, functional movement pattern evaluation, and detailed joint motion analysis of the person.

6. The method of testing and treating a person of claim 1 wherein said step of automatically generating comprises:
selecting a combination of specialized manual therapies to be used on the person by a practitioner, such as Active Release Techniques® and Muscle Activation Techniques, as well as specific corrective exercises for the person to perform to increase joint function, create stability, and improve flexibility.

7. The method of testing and treating a person of claim 1 wherein said step of automatically generating comprises:
generating a set of movement patterns designed for the correction of: postural deviation patterns; joint range-of-motion limitations; muscular subsystem imbalances;
specific muscular weakness; and functional movement compensation patterns in the person.

8. The method of testing and treating a person of claim 1 wherein said step of automatically generating comprises:
selecting specific corrective exercise movements for the person to execute to help realign posture, correct muscular imbalances, and improve movement ability.

9. The method of testing and treating a person of claim 8 wherein said step of automatically generating further comprises:
identification of the primary muscles which are being trained;
identification of the subsystem and core portion of the body; and
identification, in a step-wise fashion, of the proper performance of the corrective, and performance-based exercises.

10. The method of testing and treating a person of claim 8 wherein said step of automatically generating further comprises:
selecting specific corrective exercise movements for the person to execute to increase functional demands of targeted muscles and subsystems for improved intra-muscular and inter-muscular recruitment sequencing.

11. The method of testing and treating a person of claim 8 wherein said step of automatically generating comprises:
selecting specific corrective exercise movements for the person to execute to lengthen/stretch antagonist muscles and subsystems for improved joint movement ability.

12. The method of testing and treating a person of claim 8 wherein said step of automatically generating comprises:
selecting specific corrective exercise movements for the person to execute to implement neurological re-programming of muscles for the development of more efficient motor control and enhanced coordination of integrated joint movement patterns.

13. The method of testing and treating a person of claim 8 wherein said step of automatically generating comprises:
selecting specific corrective exercise movements for the person to execute for the benefits of improved muscular recruitment sequencing, increased joint movement ability, enhanced neurological re-programming and motor control to correct muscular imbalances, and eliminate movement pattern compensation.

14. The method of testing and treating a person of claim 1 further comprising:
defining, using a computerized database system, a set of continuing corrective, functional, and performance-based exercises, based upon the step of identifying, to cause the person to create muscle memory by replicating the proper movement or sequence of movements as a result of their body and mind being conditioned to move in the proper mode.

15. A method of using a computer system for generating person-specific data to assist a practitioner to test, train, or treat a person to improve their movement ability, the method comprising the steps of:
performing, using a computer-generated set of instructions, a postural pattern assessment, functional movement pattern evaluation, and detailed joint motion analysis of the person to identify postural deviations, discover muscular imbalances, and unveil movement limitations;
automatically identifying, using the computer and based upon the data gathered by a practitioner in the step of performing the assessments and input results to the computer, a set of specific corrective exercises and specialized manual therapies for the person to perform to increase their joint function, create stability, and improve flexibility; and
outputting data from the computer, which defines a set of continuing functional and performance-based exercises, based upon the step of identifying, to cause the person to create muscle memory by replicating the proper movement or sequence of movements as a result of their body and mind being conditioned to move in the proper mode.

16. The method of using a computer system for generating person-specific data to assist a practitioner to test, train, and treat a person of claim 15 wherein said step of performing comprises:
having a practitioner generate person-specific data by conducting an in-depth interview designed to establish fitness objectives, set realistic performance goals, and determine any specials needs or challenges that may interfere with obtaining optimal results.

17. The method of using a computer system for generating person-specific data to assist a practitioner to test, treat, and train a person of claim 16 wherein said step of performing further comprises:
having a practitioner generate person-specific data by executing a postural pattern assessment of the person, followed by a detailed joint-by-joint stability and range-of-motion analysis of the person.

18. The method of using a computer system for generating person-specific data to assist a practitioner to test, train, and treat a person of claim 17 wherein said step of performing further comprises:
automatically correlating detailed information gathered in the steps of conducting and executing with a Functional Movement Pattern Evaluation and General Exercise Readiness Test to determine the best exercise program and treatment plan for the person.

19. The method of using a computer system for generating person-specific data to assist a practitioner to test, train, and treat a person of claim 15 wherein said step of performing comprises:
having a practitioner generate person-specific data by implementing a biomechanically-based system of combined postural evaluation, movement pattern analysis, and detailed joint motion analysis of the person.

20. The method of using a computer system for generating person-specific data to assist a practitioner to test, train, and treat a person of claim 15 wherein said step of identifying comprises:
automatically selecting a combination of specialized manual therapies to be used on the person, such as Active Release Techniques® and Muscle Activation Techniques, as well as specific corrective exercises for the person to perform to increase joint function, create stability, and improve flexibility.

21. The method of using a computer system for generating person-specific data to assist a practitioner to test, train, and treat a person of claim 15 wherein said step of identifying comprises:
automatically generating a set of movement patterns designed for the correction of: postural deviation patterns; joint range-of-motion limitations; muscular subsystem imbalances; specific muscular weakness; and general movement compensation patterns in the person.

22. The method of using a computer system for generating person-specific data to assist a practitioner to test, train, and treat a person of claim 15 wherein said step of defining comprises:
automatically selecting specific corrective exercise movements for the person to execute to help realign posture, correct muscular imbalances, and improve movement ability.

23. The method of using a computer system for generating person-specific data to assist a practitioner to test, train, and treat a person of claim 22 wherein said step of defining further comprises:
identification of the primary muscles which are being trained;
identification of the subsystem and core portion of the body; and
identification, in a step-wise fashion, of the proper performance of the functional and performance-based exercises.

24. The method of using a computer system for generating person-specific data to assist a practitioner to test, train, and treat a person of claim 22 wherein said step of defining further comprises:
automatically selecting specific corrective exercise movements for the person to execute to increase functional demands of targeted muscles and subsystems for improved intra-muscular and inter-muscular recruitment sequencing.

25. The method of using a computer system for generating person-specific data to assist a practitioner to test, train, and treat a person of claim 22 wherein said step of defining comprises:
automatically selecting specific corrective and performance exercise movements for the person to execute to lengthen/stretch antagonist muscles and subsystems for improved joint movement ability.

26. The method of using a computer system for generating person-specific data to assist a practitioner to test, train, and treat a person of claim 22 wherein said step of defining comprises:
automatically selecting specific corrective exercise movements for the person to execute to implement neurological re-programming of muscles for the development of more efficient motor control and enhanced coordination of integrated joint movement patterns.

27. The method of using a computer system for generating person-specific data to assist a practitioner to test, train, and treat a person of claim 22 wherein said step of defining comprises:
automatically selecting specific corrective exercise movements for the person to execute to improve muscular recruitment sequencing, increase joint movement ability, enhance neurological re-programming and motor control to correct muscular imbalances, and eliminate movement pattern compensation.
